(19) United States
(12) Patent Application Publication
(10) Pub. No.: US 2007/0292502 A1
(43) Pub. Date: Dec. 20, 2007

(54) Chitosan-Embedded or Encapsulated Capsule

(75) Inventors: William T.H. Chang, Taipei (TW); James H.Y. Chen, Taipei (TW)

Correspondence Address: LADAS & PARRY, 26 WEST 61ST STREET, NEW YORK, NY, 10023, US

(73) Assignee: LYTONE ENTERPRISE INC., Taipei (TW)

(21) Appl. No.: 10/593,670

(22) PCT Filed: Mar. 23, 2004
(86) PCT No.: PCT/US04/08768

§ 371 Date: Apr. 24, 2007

(51) Int. Cl. A61K 9/48; A61K 31/715; A61P 3/00

(52) U.S. Cl. 424/463

(57) ABSTRACT

The present invention relates to a chitosan-embedded or encapsulated capsule, which comprises a chitosan content which is chitosan, a salt thereof, or a derivative thereof, and an edible gum embedding or encapsulating the content.

### FIELD OF THE INVENTION

The present invention relates to a chitosan-embedded or encapsulated capsule, which comprises a chitosan content, which is chitosan, a salt thereof, or a derivative thereof, and an edible gum embedding or encapsulating the content.

### BACKGROUND OF THE INVENTION

Chitosan is a polysaccharide composed of (1→4)-linked 2-acetamido-2-deoxy-β-D-glucopyranosyl residues, and is produced by fully or partially deacetylating chitin.

Products containing chitosan of various molecular weights, e.g. 10 to 1,000 kDa, are commercially available. The deacetylation degree of the chitosan content of commercial products is in general 70% to 90%. Chitosan is widely used in cosmetics, foods, nutrition supplements and pharmaceuticals. Chitosan has been shown to be capable of interfering the absorption of lipids. See, for instance, Kanauchi et al., Biosci. Biotechnol. Biochem., 59(5), 786-790 (1995). Chitosan can hardly be digested. It is capable of dissolving and adsorbing lipids and cholesterol, and is thus used for reducing body weight and cholesterol.

For food and pharmaceutical applications, chitosan is mainly used in an anhydrous state, e.g., in the form of a tablet and capsule. Conventional chitosan and modified chitosan is sparsely soluble. Its water retention and bioavailability is thus limited. U.S. Pat. No. 6,638,918 discloses modified chitosan with a better solubility and bioavailability.

When dissolved in an acidic solution, chitosan is positively charged; therefore it may be hardly compatible with other ingredients in the solution and may result in the problem of instability. The positive charge of chitosan also results in a strong mouth-puckering taste. The forms and applications of foods and pharmaceuticals comprising chitosan are thus limited. U.S. Pat. No. 6,458,938 discloses a conjugate of chitosan and polypropylene glycol, which has an improved solubility and compatibility with other compounds.

Nonetheless, up to now, no prior art reference has ever disclosed a chitosan product which does not have the mouth-puckering taste of chitosan but also keeps a lipid absorbing activity and where applicable, other desired functions, or a method for achieving such products.

### SUMMARY OF THE INVENTION

The present invention relates to a chitosan embedded or encapsulated capsule, which comprises a chitosan content which is chitosan, a salt thereof, or a derivative thereof, and an edible gum embedding or encapsulating the chitosan content.

### DETAILED DESCRIPTION OF THE INVENTION

It is surprisingly found that the above-mentioned problems of chitosan, i.e., the mouth-puckering taste, the low solubility in water solutions, and the instability and incompatibility with other compounds thus resulted, can be solved by embedding or encapsulating it with an edible gum.

The invention thus provides a chitosan embedded or encapsulated capsule, which comprises a chitosan content which is chitosan, a salt thereof, or a derivative thereof, and an edible gum embedding or encapsulating the content.

The “chitosan-embedded capsule” of the invention refers to a capsule wherein the chitosan content is mixed with or embedded in an edible gum. The “chitosan-encapsulated capsule” of the invention refers to a capsule wherein the chitosan content is coated or encapsulated with an edible gum.

### Chitosan Content

All forms of chitosan are suitable for use in the invention. For instance, chitosan can be produced by deacetylating polysaccharide chitin derived from the biomass of shellfish such as shrimp and crab, squid, an insect source, or a fungal source, and isolating and purifying the resultant products. Commercial products of various degrees of deacetylation (the number of free amino groups), purity, molecular weight distribution and viscosity can also be used in the invention.

In accordance with the invention, the salt of chitosan can be a salt formed between chitosan and an organic or inorganic acid. Examples of organic acid salts of chitosan include, but not limited to, chitosan methylate, chitosan ethylate, chitosan propylate, chitosan chloroethylate, chitosan hydroxyethylate, chitosan butylate, chitosan isobutylate, chitosan propenylate, chitosan citrate, chitosan tartrate, and the mixture thereof. Examples of inorganic acid salts of chitosan include, but not limited to, chitosan hydrochloride, chitosan hydrobromide, chitosan phosphorate and the mixture thereof.

Chitosan derivatives suitable for use in the invention include, but not limited to, a middle or long chain N-alkyl or N-alkanoyl chitosan. The term “a middle chain N-alkyl or N-alkanoyl” used herein refers to an N-alkyl or N-alkanoyl group containing 8 to 13 carbon atoms. The term “a long chain N-alkyl or N-alkanoyl” used herein refers to an N-alkyl or N-alkanoyl group containing 14 to 18 carbon atoms.

It is known that the effect of chitosan on lipid digestion and absorption increases along with the degree of deacetylation thereof. See Deuchi, K. et al., 1995, Biosci. Biotechnol. Biochem., 59(5), 781-785. In accordance with one embodiment of the invention, the degree of deacetylation of the chitosan content of the capsule of the invention is 75% or more, preferably 90% or more, and most preferably 95% or more.

In accordance with another embodiment of the invention, the molecular weight of the chitosan moiety of the chitosan content of the capsule of the invention is 1,000 to 1,000,000, preferably 30,000 to 300,000, and most preferably 100,000 to 200,000.

In accordance with a further embodiment of the invention, the average particle size of the chitosan content of the capsule of the invention is less than 40 to 50 meshes, preferably less than 60 to 100 meshes, and most preferably less than 100 to 150 meshes.

In accordance with an even further embodiment of the invention, the chitosan moiety in the chitosan content of the capsule of the invention comprise, on the basis of the weight of the embedding edible gum solutions, 0.5% to 30%, preferably 5% to 15%.

### Edible Gums

Any edible gums, reversible or irreversible, can be used in the invention to embed the chitosan content. Examples of edible gums suitable for use in the invention include, but not limited to, locust bean gum, agar gel, sodium alginate, Konjac gum, guar gum, gum Arabic, carrageenan, xanthan gum, pectin, tragacanth gum, gelatin, and the mixture thereof.

In response to multivalent metal ion (such as Ca²⁺) exchanges, or to the change in pH value or temperature, edible gums, which contain glycosides, will form insoluble gels through a cross linking reaction. In accordance with the invention, edible gums in an acidic form or in a form containing a metal ion can also be used. In addition, other synthetic or improved materials having similar properties to the above-described edible gums can also be used in the invention.

In accordance with an embodiment of the invention, the edible gum is preferably sodium alginate, guar gum, gum Arabic, carrageenan, xanthan gum, pectin, tragacanth gum or the mixture thereof. More preferably, the edible gum is sodium alginate, guar gum, gum Arabic and carrageenan, or the mixture thereof. Most preferably, the edible gum is sodium alginate. Sodium alginate is not toxic. It is a processed product of the extract of edible algae, and is widely used in food industry as viscosity enhancing agent and stabilizer. It is also as the main ingredient of analogue bionical food. Moreover, sodium alginate reacts with calcium to form a gel of Ca-alginate, which is highly heat- and acid-stable, and has a high intestinal solubility.

According to an embodiment of the invention, the edible gum comprises 0.5% to 30.0% by wet weight of the capsule of the invention, preferably 1.0% to 2.0%.

According to another embodiment of the invention, the chitosan embedded or encapsulated capsule comprises sodium alginate and gelatin or Konjac gum. The amount of gelatin or Konjac gum, if added, comprises 0.3% to 6% by weight of the edible gum.

### Other Components

To improve the values, varieties, flavors and palatability, the chitosan embedded or encapsulated capsule of the invention may favorably comprises health enhancing components, and/or general food additives, such as flavoring agents, sweetening agents, coloring agents, other additives that may adjust or improve palatability, and the mixture thereof.

Any components that may enhance health can be embedded or encapsulated in the capsule together with chitosan. Examples of such components include, but not limited to, red yeast (Monascus), blue algae, green algae, green tea, blue berry, mushroom, Ganoderma lucidum, Cordyceps sinensis, Antrodia camphorata, Ginseng, lactic acid bacterial, Bifidobacteria, cellulose and the mixture thereof. These components can be added in the form of dry pulverized extract powder, liquid, or semi-liquid (e.g., paste), and in the amount of 0.1% to 10% by wet weight of the capsule.

The flavoring agents used in the invention can be any natural or synthetic flavoring agents that provide a smell of fruits, flowers or nuts, including, but not limited to, a smell of apples, strawberries, lemons, oranges, grapes, cherries, peaches, cocoa, teas, roses, jasmine, sweet osmanthus, almonds and the like.

The coloring agents used in the invention can be any natural or synthetic coloring agents. Examples for coloring agents suitable for use in the invention include, but not limited to, Gardenia, beet root, Lycopene, Perilla colors, Tartrazine, cochineal Red A, and the like.

The sweetening agents used in the invention can be any natural or synthetic sweetening agents, including, but not limited to, sucrose, fructose, glucose, stevioside and aspartame.

### Preparation of Chitosan Embedded or Encapsulated Capsule

Conventional methods of gelation can be used to embed or encapsulate the chitosan content of the chitosan capsule of the invention with a suitable edible gum. In one embodiment of the invention, the capsule of the invention is prepared by mixing the chitosan content and edible gum, and coagulating the resultant mixture in accordance with the mode of gelation of the edible gum. For instance, U.S. Pat. No. 4,692,284 and U.S. Pat. No. 5,472,648 disclose polymeric materials exhibiting gelling properties, such as sodium alginate, and the large-scale production of capsules comprising the materials. The patents in their entire contents are incorporated herewith as references of the invention.

In another embodiment of the invention, the capsule of the invention is prepared by encapsulating the chitosan content with the edible gum.

### Utility of Chitosan Embedded Capsule

Embedding or encapsulating chitosan with an edible gum in accordance with the invention can effectively solve the mouth-puckering taste of chitosan present in foods in a liquid form. Embedding or encapsulated chitosan with an edible gum in accordance with the invention also significantly increases the amount of chitosan in liquid foods up to 20% by weight, but does not result in the unpleasant taste or cause a problem in viscosity or stability after chitosan is dissolved. The chitosan-embedded or encapsulated capsule of the invention breaks through the application limitations of chitosan due to its properties, and can be used in food products containing a high percentage of water, such as jelly, pudding, and confectionery. The chitosan-embedded or encapsulated capsule of the invention can avoid the problems caused by adding chitosan in a powder or liquid form, such as the mouth-puckering taste, poor palatability and appearance, and instability. It also increases the amount of the chitosan content of food products. Furthermore, the capsule of the invention may comprise additives such as health enhancing components, flavoring agents, and coloring agents, which increase the values, varieties, compliance and convenience of products containing chitosan.

### EXAMPLES

### Example 1

### Preparation of Chitosan-Sodium Alginate Capsules

The example illustrates the general method for preparing the chitosan embedded capsule of the invention.

A 1% sodium alginate solution was prepared. After the sodium alginate was completely dissolved through strong stirring, 20 gm of chitosan powder (Premix, FG95 ChitoClear) was added to and blended with 200 ml of the 1% sodium alginate solution. The resultant mixture was put into a 50-ml syringe with a 0.8 mm needle. Capsules of chitosan-sodium alginate mixture were produced by pushing the mixture out of the syringe and into a 1.5% CaCl₂ solution. Capsules of an average diameter of ca. 2.0 mm were formed and cured via ion-exchange of Ca²⁺. The chitosan-sodium alginate capsules were allowed to stay in the CaCl₂ solution for 30 min., and washed with distilled water. The resultant capsules can directly be used in beverage and other foods. Alternatively, the chitosan-sodium alginate capsules can be dehydrated to form dried products, and reconstituted with water right before use.

### Example 2

### Preparation of Red Yeast-Chitosan Capsules

This example illustrates the preparation of capsules comprising red yeast and chitosan.

Capsules were prepared in a way similar to that described in Example 1, except that, in addition to 20 gm of chitosan, 2 gm of dry red yeast powder was added, with stirring, to 200 ml of 1% sodium alginate solution to produce a sodium alginate solution comprising chitosan and red yeast. In a way similar to that described in Example 1, the solution was put into and then pushed through a syringe. The resultant capsules were dropped into a 1.5% CaCl₂ solution to be cured. After washing and drying, capsules comprising red yeast and chitosan were produced.

In a way similar to that described in this example, other additives, such as green algae powder, green tea powder, flavoring agents, coloring agents and sweetening agents, can be added to the capsules of the invention. The amount of these additives can be adjusted as desired. In general, the amount of the optional additives ranges from 0.1% to 10% of the weight of the sodium alginate solution.

### Example 3

### Sensory Evaluation of Chitosan Embedded Capsules

A sensory evaluation was performed to compare the mouth-puckering taste of the chitosan embedded capsules prepared in Example 1 and unembedded chitosan powder. The unembedded chitosan powder and chitosan embedded capsules were added to sports drink, juice or yogurt. The products were randomly evaluated by five persons. The score corresponding to the puckering taste is as follows: 1=no taste, 2=very weak, 3=weak, 4=medium, 5=slightly strong, 6=strong and 7=very strong. The average scores of the chitosan embedded capsules and the unembedded chitosan powder were 1.4 and 6.8, respectively.

The test results show that the chitosan-sodium alginate capsules can effectively reduce the puckering taste caused by chitosan.

1. A chitosan embedded or encapsulated capsule, comprising a chitosan content which is a chitosan, a salt thereof or a derivative thereof, and an edible gum embedding the chitosan content.

2. The chitosan embedded or encapsulated capsule of claim 1, wherein the deacetylation degree of the chitosan content is 75% or more.

3. The chitosan embedded or encapsulated capsule of claim 2, wherein the deacetylation degree of the chitosan content is 90% or more.

4. The chitosan embedded or encapsulated capsule of claim 3, wherein the deacetylation degree of the chitosan content is 95% or more.

5. The chitosan embedded or encapsulated capsule of claim 1, wherein the molecular weight of the chitosan moiety of the chitosan content is 1,000 to 1,000,000.

6. The chitosan embedded or encapsulated capsule of claim 5, wherein the molecular weight of the chitosan moiety of the chitosan content is 30,000 to 300,000.

7. The chitosan embedded or encapsulated capsule of claim 6, wherein the molecular weight of the chitosan moiety of the chitosan content is 100,000 to 200,000.

8. The chitosan embedded or encapsulated capsule of claim 1, wherein the average particle size of the chitosan content is in the range of 40 to 50 meshes.

9. The chitosan embedded or encapsulated capsule of claim 8, wherein the average particle size of the chitosan content is in the range of 60 to 100 meshes.

10. The chitosan embedded or encapsulated capsule of claim 9, wherein the average diameter of the chitosan content is in the range of 100 to 150 meshes.

11. The chitosan embedded or encapsulated capsule of claim 1, wherein the amount of the chitosan content is 0.5% to 30% by wet weight of the capsule.

12. The chitosan embedded or encapsulated capsule of claim 11, wherein the amount of the chitosan content is 5% to 15% by wet weight of the capsule.

13. The chitosan embedded or encapsulated capsule of claim 1, wherein the salt of chitosan is an inorganic salt selected from the group consisting of chitosan hydrochloride, chitosan hydrobromide, chitosan phosphorate, and the mixture, or an organic salt selected from the group consisting of chitosan methylate, chitosan ethylate, chitosan propylate, chitosan chloroethylate, chitosan hydroxyethylate, chitosan butylate, chitosan isobutylate, chitosan propenylate, chitosan citrate, chitosan tartrate, and the mixture thereof.

14. The chitosan embedded or encapsulated capsule of claim 1, wherein the derivative of chitosan is C₈-C₁₈N-alkyl or C₈-C₁₈N-alkanoyl chitosan.

15. The chitosan embedded or encapsulated capsule of claim 1, wherein the edible gum is selected from the group consisting of locust bean gum, agar gel, sodium alginate, Konjac gum, guar gum, Arabic gum, carrageenan, xanthan gum, pectin, tragacanth gum, gelatin or the mixture thereof.

16. The chitosan embedded or encapsulated capsule of claim 1, wherein the edible gum is sodium alginate.

17. The chitosan embedded or encapsulated capsule of claim 1, wherein the amount of the edible gum comprises 0.5% to 5.0% by wet weight of the capsule.

18. The chitosan embedded or encapsulated capsule of claim 18, wherein the amount of the edible gum comprises 1.0% to 2.0% by wet weight of the capsule.

19. The chitosan embedded or encapsulated capsule of claim 1, wherein the edible gum further comprises, on the basis of the weight of the edible gum, 0.3% to 6% of gelatin and Konjac gum.

20. The chitosan embedded or encapsulated capsule of claim 1, further comprising health-enhancing components or food additives.

21. The chitosan embedded or encapsulated capsule of claim 1, which can effectively reduce the mouth-puckering taste of chitosan.
